(19) United States
(12) Patent Application Publication
(10) Pub. No.: US 2023/0172139 A1
(43) Pub. Date: Jun. 8, 2023

(54) CLUBROOT RESISTANCE

(71) Applicant: Cargill, Incorporated, Wayzata, MN (US)

(72) Inventors: Paul A. COVEY, Fort Collins, CO (US); Benyuan DANG, Wayzata, MN (US); Richard FLETCHER, Windsor, CO (US); Lisa Elizabeth SULLIVAN, Eaton, CO (US)

(73) Assignee: Cargill, Incorporated, Wayzata, MN (US)

(21) Appl. No.: 17/997,195

(22) PCT Filed: Apr. 30, 2021
(86) PCT No.: PCT/US2021/030067

§ 371 Date: Oct. 26, 2022

Related U.S. Application Data
(60) Provisional application No. 63/023,004, filed on May 11, 2020.

(51) Int. Cl. A01H 6/20; A01H 1/00

(52) U.S. Cl. CPC A01H 6/202; A01H 1/1245

(57) ABSTRACT

This disclosure describes clubroot (CR) resistant plants; in particular, CR resistant Brassica plants, including B. napus. CR resistant plants include all or part of at least one genomic sequence of a B. napus parent genome that confers clubroot resistance. The genomic sequence may be a genomic sequence from chromosome N03, chromosome N04, and/or chromosome N08.

### CROSS-REFERENCE TO RELATED APPLICATIONS

This application claims the benefit of U.S. Provisional Application No. 63/023,004, filed May 11, 2020, and entitled “Clubroot Resistance,” which is incorporated herein by reference in its entirety.

### TECHNICAL FIELD

This disclosure describes clubroot (CR) resistant plants; in particular, CR resistant Brassica plants, including B. napus.

### BACKGROUND OF THE INVENTION

Clubroot is a serious soil-borne disease of cruciferous plants, such as cabbage, broccoli, cauliflower, brussel sprouts, radishes, turnips, canola and other plants of the family Brassicaceae. In canola, it causes swellings or galls to form on the roots. These formations impede nutrient and water uptake and can cause plant death, wiping out important money generating canola crops. It is caused by a protist called Plasmodiophora brassicae.

Clubroot can be spread from plant to plant and through soil infested with resting spores. Infested soil can be carried from field to field by farm machinery and can also be moved by wind and water erosion. P. brassicae is able to infect 300 species of cruciferous plants, making this disease a recurring problem even with crop rotation. Further, chemical treatments to control the disease are either banned due to environmental regulations or are not cost effective.

### SUMMARY OF THE INVENTION

This disclosure is based, at least in part, on the discovery that Brassica plants can be produced which are resistant to one or more, such as several, pathotypes of clubroot (CR).

The disclosure provides a Brassicaceae or Brassica plant or a part thereof comprising all or part of at least one genomic sequence of a B. napus parent genome that confers clubroot resistance, wherein said genome sequence is selected from the group consisting of: a) the genomic sequence on chromosome N08 between nucleotide positions 10,799,175 and 11,730,703; b) the genomic sequence on chromosome N04 between nucleotide positions 16,884,111 and 20,563,734; and c) the genomic sequence on chromosome N03 between nucleotide positions 14,845,698 to 15,791,893; wherein said Brassicaceae plant has resistance to one or more pathotypes of clubroot (CR).

The said genomic sequence on chromosome N03 between nucleotide positions 14,845,698 to 15,791,893 may comprise a single nucleotide polymorphism (SNP) at a position selected from the group consisting of 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; and 15,711,283. In another aspect, said genomic sequence comprises at least five SNPs at five different positions selected from the group consisting of 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; and 15,711,283. In another aspect, said genomic sequence comprises at least 10 SNPs at 10 different positions selected from the group consisting of 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; or 15,711,283. In another aspect, said genomic sequence comprises at least 15 SNPs at 15 different positions selected from the group consisting of 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; and 15,711,283.

The genomic sequence on chromosome N04 between nucleotide positions 16,884,111 to 20,563,734 may comprise a SNP at a position selected from the group consisting of 16,548,548; 16,884,111; 18,420,558; 18,548,589; 19,307,982; 19,579,676 and/or 20,563,734. In another aspect, said genomic sequence comprises at least two SNPs at two different positions selected from the group consisting of 16,548,548; 16,884,111; 18,420,558; 18,548,589; 19,307,982; 19,579,676 and 20,563,734. In another aspect, said genomic sequence comprises at least five SNPs at five different positions selected from the group consisting of 16,548,548; 16,884,111; 18,420,558; 18,548,589; 19,307,982; 19,579,676 and 20,563,734.

The genomic sequence on chromosome N08 between nucleotide positions 10,799,175 to 11,730,703 may comprise a SNP at a position selected from the group consisting of 10,651,892; 10,661,594; 10,799,176; 11,126,290; 11,181,573; 11,481,275; 11,588,240; 11,730,703 and 11,804,795. In another aspect, said genomic sequence comprises at least two SNPs at two different positions selected from the group consisting of 10,651,892; 10,661,594; 10,799,176; 11,126,290; 11,181,573; 11,481,275; 11,588,240; 11,730,703 and 11,804,795. In another aspect, said genomic sequence comprises at least five SNPs at five different positions selected from the group consisting of 10,651,892; 10,661,594; 10,799,176; 11,126,290; 11,181,573; 11,481,275; 11,588,240; 11,730,703 and 11,804,795. In another aspect, said genomic sequence comprises at least nine SNPs at nine different positions selected from the group consisting of 10,651,892; 10,661,594; 10,799,176; 11,126,290; 11,181,573; 11,481,275; 11,588,240; 11,730,703 and 11,804,795.

The genomic sequence may comprise all or part of said genomic sequence on chromosome N03 between nucleotide positions 14,845,698 and 15,791,893 and all or part of the genomic sequence on chromosome N04 between nucleotide positions 16,884,111 and 20,563,734. In another aspect, said genomic sequence comprises all or part of said genomic sequence on chromosome N03 between nucleotide positions 14,845,698 and 15,791,893 and all or part of the genomic sequence on chromosome N08 between nucleotide positions 10,799,175 and 11,730,703. In another aspect, said genomic sequence comprises all or part of said genomic sequence on chromosome N08 between nucleotide positions 10,799,175 and 11,730,703 and all or part of the genomic sequence on chromosome N04 between nucleotide positions 16,884,111 and 20,563,734.

The genomic sequence may comprise from 25 to 50, 25 to 100, 50 to 200, 100 to 500, 250 to 1,000, 500 to 5,000, 2,000 to 10,000, 5,000 to 20,000, 10,000 to 100,000, 50,000 to 400,000, 25,000 to 1,000,000, 100,000 to 1,000,000, 200,000 to 1,000,000, or 500 to 1,000,000 contiguous nucleotides of the genomic sequence of said B. napus parent genome.

The plant may be selected from the group consisting of Camelina sativa, Brassica napus, Brassica oleracea, Brassica juncea, Brassica nigra, Brassica rapa, and Brassica carinata. In some aspects, said plant is selected from the group consisting of Brassica napus, Brassica rapa, and Brassica juncea. In some aspects, said plant is tolerant of an herbicide, such as one from the group consisting of imidazolinone, dicamba, cyclohexanedione, sulfonylurea, glyphosate, glufosinate, phenoxy propionic acid, L-phosphinothricin, triazine, and benzonitrile.

The plant may be resistant to one or more clubroot pathotypes selected from the group consisting of 2, 3, 3A, 3D, 5, 6, 8, FTC5X, LG1, LG2, LG3, 3H, 2B, 5X, C4W.

The resistance traits described herein can also be stacked with other known or available clubroot resistance traits, such as those found in the varieties sold under the trade name VICTORY HYBRID CANOLA and summarized on the World Wide Web at cargill.ca (in U.S. Provisional Application No. 63/023,004, this information was referenced by a link to the World Wide Web at cargillag.ca/Media/Default/PDFs/19GEOS11411%20-%20VICTORY%202020%20Program% 20Guide-v7web.pdf). Said varieties include, but are not limited to, V14-1, V12-3, V24-1, and/or V25-1T.

### DETAILED DESCRIPTION OF THE INVENTION

Clubroot (CR) continues to spread, posing a threat to canola production. Cultivar resistance (CR resistant cultivars) is one method of clubroot management. As described herein, Brassica plants can be produced with resistance to one or more CR pathotypes, including, but not limited to, pathotypes 2, 3, 3A, 3D, 5, 6, 8, FTC5X, LG1, LG2, LG3, 3H, 2B, 5X, C4W.

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular aspects only and is not intended to be limiting of the invention.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the term “about” means plus or minus 10% of the indicated value. For example, about 100 means from 90 to 110.

As used herein, the terms “including”, “includes”, “having”, “has”, “with”, or variants thereof, are intended to be inclusive similar to the term “comprising.”

As used herein, said “contain”, “have” or “including” include “comprising”, “mainly consist of”, “basically consist of” and “formed of”; “primarily consist of”, “generally consist of” and “comprising of” belong to generic concept of “have” “include” or “contain.”

In some aspects, a plant provided herein is a plant found in the “Triangle of U”, i.e. a plant of genus Brassica: Brassica napus (AA CC genome; n=19), which is an amphidiploid plant of the Brassica genus, but is thought to have resulted from hybridization of Brassica rapa (AA genome; n=10) and Brassica oleracea (CC genome; n=9). Brassica juncea (AA BB genome; n=18) is an amphidiploid plant of the Brassica genus that is generally thought to have resulted from the hybridization of Brassica rapa and Brassica nigra (BB genome; n=8). Under some growing conditions, B. juncea may have certain superior traits to B. napus. These superior traits may include higher yield, better drought and heat tolerance and better disease resistance. Brassica carinata (BB CC genome; n=17) is an amphidiploid plant of the Brassica genus but is thought to have resulted from hybridization of Brassica nigra and Brassica oleracea.

In some aspects, the Brassica plant provided herein is a “canola” plant. Canola herein generally refers to plants of Brassica species that have less than 2% (e.g., less than 1%, 0.5%, 0.2% or 0.1%) erucic acid (delta 13-22:1) by weight in seed oil and less than about 30 micromoles (e.g., less than 30, 25, 20 15, or 10 micromoles) of glucosinolates per gram of oil free meal (meal fraction). Typically, canola oil may include saturated fatty acids known as palmitic acid and stearic acid, a monounsaturated fatty acid known as oleic acid, and polyunsaturated fatty acids known as linoleic acid and linolenic acid. Canola oil may contain less than about 7%(w/w) total saturated fatty acids (mostly palmitic acid and stearic acid) and greater than 40%(w/w) oleic acid (as percentages of total fatty acids). Traditionally, canola crops include varieties of Brassica napus and Brassica rapa. Non-limiting exemplary Brassica plants of the present disclosure are spring canola (Brassica napus subsp. oleifera var. annua) and winter canola (Brassica napus subsp. oleifera var. biennis). Furthermore, a canola quality Brassica juncea variety, which has oil and meal qualities similar to other canola types, has been added to the canola crop family (U.S. Pat. Nos. 6,303,849; 7,423,198; all of which are incorporated herein by reference). Likewise, it is possible to establish canola quality B. carinata varieties by crossing canola quality variants of Brassica napus with Brassica nigra and appropriately selecting progeny thereof, optionally after further backcrossing with B. carinata, B. napus, and/or B. nigra.

In some aspects, the plant provided herein is a plant in the Brassicaceae family that is a natural oilseed plant, e.g., Camelina sativa.

In some aspects, the Brassica plants provided herein can be a Brassica plant line. The term “line” refers to a group of plants that displays little to no genetic variation for at least one trait among individuals sharing that designation.

The Brassica plants and seeds disclosed herein are, in some aspects, of a species comprising a genome of one or two members of the species Brassica oleracea, Brassica nigra, and Brassica rapa. In some aspects, the Brassicaceae or Brassica plants and seeds disclosed herein are of the species Camelina sativa, Brassica napus, Brassica carinata, Brassica juncea, Brassica oleracea, Brassica nigra, or Brassica rapa. In some aspects, the plants and seeds are of the species Brassica napus and/or Brassica carinata.

In some aspects, the parent plant can have all or part of at least one genomic sequence of a B. napus parent genome that confers clubroot (CR) resistance, wherein the genomic sequence is selected from the group consisting of a) the genomic sequence on N08 between nucleotide positions 10,799,175 to 11,730,703; b) the genomic sequence on chromosome N04 between nucleotide positions 16,884,111 to 20,563,734; and c) the genomic sequence on chromosome N03 between nucleotide positions 14,845,698 to 15,791,893. In the present disclosure, nucleotide positions within a given chromosome are based on the position in the genomic sequence of Brassica napus cultivar DH12075.

In some aspects, the genomic sequence of a B. napus parent genome that confers CR resistance can include, for example, from 25 to 50, 25 to 100, 50 to 200, 100 to 500, 250 to 1,000, 500 to 5,000, 2,000 to 10,000, 5,000 to 20,000, 10,000 to 100,000, 50,000 to 400,000, 25,000 to 1,000,000, 100,000 to 1,000,000, 200,000 to 1,000,000, or 500 to 1,000,000 contiguous nucleotides or longer of a region of chromosome N08 (e.g., the genomic sequence on chromosome N08 between nucleotide positions 10,799,175 to 11,730,703), a region of the chromosome N04 (e.g., the genomic sequence on chromosome N04 between nucleotide positions 16,884,111 to 20,563,734) and/or a region of chromosome N03 (e.g., the genomic sequence on chromosome N03 between nucleotide positions 14,845,698 to 15,791,893).

In some aspects, one or more single nucleotide polymorphisms (SNPs) can be present in all or part of at least one genomic sequence of a B. napus parent genome that confers CR resistance. The presence of one or more such SNPs can be used in selecting suitable parents and progeny. A SNP can occur within coding and non-coding regions, including exons, introns, and untranslated sequences. Examples of SNPs include substitutions of one or more nucleotides, deletions of one or more nucleotides, and insertions of one or more nucleotides. In some aspects, a nucleotide substitution can be a transition, in which a purine nucleotide is substituted for another purine (e.g., A to G or G to A), or a pyrimidine nucleotide is substituted for another pyrimidine (e.g., C to T or T to C). In some aspects, a nucleotide substitution can be a transversion, in which a purine nucleotide is substituted for a pyrimidine or a pyrimidine nucleotide is substituted for a purine nucleotide (e.g., G to T, or C to G). A nucleotide substitution within a coding sequence that results in the substitution of an amino acid also can be referred to as a non-synonymous SNP.

In some aspects, a Brassica plant can include all or part of the genomic sequence on chromosome N03 between nucleotide positions 14,845,698 to 15,791,893 that confers CR resistance. In some aspects, the genomic sequence that confers CR resistance can include one or more SNPs (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more different SNPs) between nucleotide positions 14,845,698 to 15,791,893 on chromosome N03. Table 1 provides examples of SNPs within chromosome N03 that are distributed throughout the genomic sequence between or around nucleotide positions 14,845,698 to 15,791,893, including SNPs at positions 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; and/or 15,711,283.

In some aspects, a Brassica plant can include all or part of the genomic sequence on chromosome N04 between nucleotide positions 16,884,111 to 20,563,734 that confers CR resistance. In some aspects, the genomic sequence that confers CR resistance can include one or more SNPs (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more different SNPs) between nucleotide positions 16,884,111 to 20,563,734 on chromosome N04. Table 1 provides examples of SNPs within chromosome N04 that are distributed throughout the genomic sequence between or around nucleotide positions 16,884,111 to 20,563,734, including SNPs at positions 16,548,548; 16,884,111; 18,420,558; 18,548,589; 19,307,982; 19,579,676 and/or 20,563,734.

In some aspects, a Brassica plant can include all or part of the genomic sequence on chromosome N08 between nucleotide positions 10,799,175 to 11,730,703 that confers CR resistance. In some aspects, the genomic sequence that confers CR resistance can include one or more SNPs (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more different SNPs) between nucleotide positions 10,799,175 to 11,730,703 on chromosome N08. Table 1 provides examples of SNPs within chromosome N08 that are distributed throughout the genomic sequence between or around nucleotide positions 10,799,175 to 11,730,703, including SNPs at positions 10,651,892; 10,661,594; 10,799,176; 11,126,290; 11,181,573; 11,481,275; 11,588,240; 11,730,703 and/or 11,804,795.

In some aspects, a Brassica plant can include all or part of the genomic sequence on chromosome N03 between nucleotide positions 14,845,698 to 15,791,893 that confers CR resistance and all or part of the genomic sequence on chromosome N04 between nucleotide positions 16,884,111 to 20,563,734 that confers clubroot resistance. Examples of SNPs that can be found in each of these regions are described above.

In some aspects, a Brassica plant can include all or part of the genomic sequence on chromosome N03 between nucleotide positions 14,845,698 to 15,791,893 that confers CR resistance and all or part of the genomic sequence on chromosome N08 between nucleotide positions 10,799,175 to 11,730,703 that confers clubroot resistance. Examples of SNPs that can be found in each of these regions are described above.

In some aspects, a Brassica plant can include all or part of the genomic sequence on chromosome N08 between nucleotide positions 10,799,175 to 11,730,703 that confers clubroot resistance and all or part of the genomic sequence on chromosome N04 between nucleotide positions 16,884,111 to 20,563,734 that confers clubroot resistance. Examples of SNPs that can be found in each of these regions are described above.

The present disclosure also relates to oil comprising a polyunsaturated fatty acid obtainable from the plants described herein. The term “oil” refers to a fatty acid mixture comprising unsaturated and/or saturated fatty acids which are esterified to triglycerides. In some aspects, the Brassica plants are canola plants, wherein one can extract canola oil from the seeds.

In some aspects, a Brassica plant provided herein is tolerant of an herbicide such as an imidazolinone, dicamba, cyclohexanedione, a sulfonylurea, glyphosate, glufosinate, phenoxy propionic acid, L-phosphinothricin, a triazolinone, a triazolpyrimidine, a pyrimidinylthiobenzoate, and benzonitrile. For example, Brassica plants can include a polynucleotide that encodes a product (e.g., a mutant acetohydroxyacid synthase) that confers resistance to an herbicide (e.g., an imidazolinones, a sulfonylureas, a pyrimidinylthiobenzoate, a triazolinone, or a triazolopyrimidine). See, for example, Tans et al., Pest Manag Sci. 61(3):246-57 (2005) and Hu et al., PLoS One. 12(9): e0184917 (2017).

In some aspects, the plants, such as the progeny, can be hybrids or inbreds. The term hybrid relates to a cultivar or plant-breeding progeny based upon the controlled cross-pollination between or among distinct parent lines, so that the resulting seed inherits its genetic composition from those parent lines. Seed for a particular hybrid can be repeatedly and predictably produced when repeatedly making controlled cross-pollinations from the same stable female and male parent genotypes. While inbred refers to a relatively stable plant genotype resulting from doubled haploids, successive generations of controlled self-pollination, successive generations of controlled backcrossing to a recurrent parent, or other method to develop homozygosity. Backcrossing refers to a process in which a breeder repeatedly crosses hybrid progeny back to one of the parents; for example, a first-generation hybrid F1 crossed back to one of the parental genotypes of the F1 hybrid. The production of hybrid plants is well known/available to an art worker.

### EXAMPLES

The following examples are provided in order to demonstrate and further illustrate certain embodiments and aspects of the present invention and are not to be construed as limiting the scope of the invention described herein or in the claims.

### Clubroot Resistant Plants

Gry is publicly available. The spring x winter is an example of a technology to allow one to get the genes responsible for a trait into canola (U.S. Pat. No. 9,332,723; AU2015202535B2). Wichita is a publicly available variety.

Norway, N08

The Brassica napus swede variety Gry, USDA PI 443015, was planted and named [3P]Swede-01. After vernalization to break dormancy, it was crossed with the spring by winter Wichita line (US 9,332,723; AU2015202535B2; incorporated here by reference), BC5S1-Wichita, to produce F1 generation seed (CRR-napus1-01). The F1 seed was then planted and selections were made based on spring growth habit. Three plants were self-pollinated to produce an F2 population. Entry two (BC1_Sprinter Wichita×Norway2) was planted and phenotyped for resistance to Plasmodiophora brassicae, clubroot, pathotype 3, P3, and the backcross (BC) one seed was produced, spring by winter Wichita*2/Norway2. It was planted and backcrossed with a spring by winter Wichita line, BC10S3-S-Wichita1, to produce the BC2 generation, 12CRRX010. 12CRRX010 was planted and once again was phenotyped with a selection made based on resistance to P3. This selection was crossed with spring by winter Wichita to produce the BC3 generation, 12CRRX020. 12CRRX020 was planted with selections made based on resistance to P3 and crossed with spring by winter Wichita to produce BC4, 12CRRX028. This seed was planted and phenotyped with P3. A selection was made and crossed with spring by winter Wichita to produce the backcross BC5, 13CRRX012. Self-pollinated seed from this generation, BC6—self (S) one, was used to select a line with fixed resistance to clubroot. These selections, 13CRRX012.1007.013.001.015 and 13CRRX012.1007.013.001.021 with the pedigree spring by winter Wichita*5/Norway2, were used as a donor for trait introgression. They are also used as the “Norway” trait control for phenotype and genotype assays. Fine mapping and resistance testing were undertaken (Tables 1 and 2).

Iceland, N03

Brassica napus swede variety Gry, USDA PI 443015 (Norway), was planted and then vernalized to break dormancy. This line was crossed with a spring by winter Wichita, Wichita*10/fast rapa (U.S. Pat. No. 9,332,723; AU2015202535B2; incorporated here by reference), to produce F1 generation seed to produce 13CRRX040 (15CB7501.00), Wichita*10/fast rapa//[3P]Norway. 15CB7501.00 was used to produce a doubled haploid (DH) population. Once the self-pollinated DH plants were harvested the lines were phenotyped for resistance to Plasmodiophora brassicae, clubroot, pathotype Fort Collins 5X (FTC5X). Lines that were resistant were selected and those with a spring growth habit were crossed with a spring by winter Wichita, BC10S3 sWichita Rf, to produce F1 generation, BC10S3 sWichita Rf///Wichita*10/fast rapa//Norway. These F1 generations were planted and selections were made based on resistance to FTC5X and the wild type marker call for the Norway N08 trait, to ensure that a different trait was selected. The DH line 15CB7501.DH.0354 was selected as the donor for the Iceland trait. Fine mapping and resistance testing were undertaken (Tables 1 and 2).

Finland, N04

Brassica napus swede variety Gry, USDA PI 443015 (Norway), was planted and then vernalized to break dormancy. This line was crossed with a spring by winter Wichita, Wichita*10/fast rapa (U.S. Pat. No. 9,332,723; AU2015202535B2; incorporated here by reference), to produce F1 generation seed to produce 13CRRX040 (15CB7501.00), Wichita*10/fast rapa//[3P]Norway. 15CB7501.00 was used to produce a doubled haploid (DH) population. Once the self-pollinated DH plants were harvested the lines were phenotyped for resistance to Plasmodiophora brassicae, clubroot, pathotype Fort Collins 5X (FTC5X). Lines that were resistant were selected and those with a spring growth habit were crossed with a spring by winter Wichita, BC10S3 sWichita Rf, to produce F1 generation, BC10S3 sWichita Rf///Wichita*10/fast rapa//Norway. These F1 generations were planted and selections were made based on resistance to FTC5X and the wild type marker call for the Norway N08 trait. The DH line 15CB7501.DH.0354 was selected as the donor for the Iceland trait. During the mapping of the N03 Iceland trait, it was found that a second clubroot resistance trait was present on the N04 chromosome in the 15CB7501.DH.0354 donor. By allowing the lines to segregate during self-pollination to an F4 generation, an entry that is wild type for Iceland yet homozygous for the resistance trait on N04 was identified. This trait was given the name Finland. Fine mapping and resistance testing were undertaken (Tables 1 and 2).

TABLE 1
Fine Mapping of Iceland, Finland and Norway

| | | | Het - | Het - | | | Het - | Het - | | | | |
|---|---|---|---|---|---|---|---|---|---|---|---|---|
| | | | Incom- | Incom- | | | Incom- | Incom- | | Path- | | |
| | Wild | Vari- | plete | plete | Wild | Vari- | plete | plete | | ogen - | | |
| | Type - | ant - | Domi- | Domi- | Type - | ant - | Domi- | Domi- | | Club- | Chro- | |
| | Suscep- | Resis- | nance, | nance, | Suscep- | Resis- | nance, | nance, | | root | mo- | DH12075 |
| Marker | tible | tant | some | some | tible | tant | some | some | Trait | (CRR) | some | Location |
| Bn-A03-p10458923 | C:C | T:T | C:T | T:C | 0 | 2 | 1 | 1 | Iceland | CRR | N03 | 10,708,311 |
| Bn-A03-p11744082 | T:T | G:G | G:T | T:G | 0 | 2 | 1 | 1 | Iceland | CRR | N03 | 12,061,079 |
| Bn_N3_Iceland_p11957752 | C:C | G:G | C:G | G:C | 0 | 2 | 1 | 1 | Iceland | CRR | N03 | 12,279,028 |
| Bn-A03-p12279241 | A:A | G:G | G:A | A:G | 0 | 2 | 1 | 1 | Iceland | CRR | N03 | 12,629,150 |
| Bn_N3_Iceland_p13271907 | G:G | A:A | G:A | A:G | 0 | 2 | 1 | 1 | Iceland | CRR | N03 | 13,606,968 |
| Bn_N3_Iceland_p13281917 | G:G | A:A | G:A | A:G | 0 | 2 | 1 | 1 | Iceland | CRR | N03 | 13,616,978 |
| Bn-A03-p13465062 | C:C | T:T | C:T | T:C | 0 | 2 | 1 | 1 | Iceland | CRR | N03 | 13,787,184 |
| Bn-A03-p14176469 | G:G | A:A | G:A | A:G | 0 | 2 | 1 | 1 | Iceland | CRR | N03 | 14,509,547 |
| Bn-A03-p14306440 | A:A | C:C | C:A | A:C | 0 | 2 | 1 | 1 | Iceland | CRR | N03 | 14,641,501 |
| Bn-A03-p14353080 | G:G | T:T | G:T | T:G | 0 | 2 | 1 | 1 | Iceland | CRR | N03 | 14,687,684 |
| Bn-A03-p14369287 | T:T | G:G | G:T | T:G | 0 | 2 | 1 | 1 | Iceland | CRR | N03 | 14,693,565 |
| Bn-A03-p14423477 | A:A | G:G | G:A | A:G | 0 | 2 | 1 | 1 | Iceland | CRR | N03 | 14,752,111 |
| Bn-A03-p14473296 | C:C | T:T | C:T | T:C | 0 | 2 | 1 | 1 | Iceland | CRR | N03 | 14,805,303 |
| Bn-A03-p14511431 | G:G | A:A | G:A | A:G | 0 | 2 | 1 | 1 | Iceland | CRR | N03 | 14,845,698 |
| Bn-A03-p15002477 | A:A | G:G | G:A | A:G | 0 | 2 | 1 | 1 | Iceland | CRR | N03 | 15,359,664 |
| Bn_N3_Iceland_p15103452 | G:G | T:T | G:T | T:G | 0 | 2 | 1 | 1 | Iceland | CRR | N03 | 15,461,403 |
| Bn_N3_Iceland_p15336662 | T:T | C:C | T:C | C:T | 0 | 2 | 1 | 1 | Iceland | CRR | N03 | 15,694,613 |
| Bn_N3_Iceland_p15224191 | C:C | T:T | C:T | T:C | 0 | 2 | 1 | 1 | Iceland | CRR | N03 | 15,697,299 |
| Bn_N3_Iceland_p14392932 | A:A | G:G | A:G | G:A | 0 | 2 | 1 | 1 | Iceland | CRR | N03 | 15,711,283 |
| Bn-A04-p14582584 | A:A | C:C | C:A | A:C | 0 | 2 | 1 | 1 | Finland | CRR | N04 | 16,548,548 |
| Bn-A04-p14917114 | G:G | T:T | G:T | T:G | 0 | 2 | 1 | 1 | Finland | CRR | N04 | 16,884,111 |
| Bn-A04-p16445225 | G:G | A:A | G:A | A:G | 0 | 2 | 1 | 1 | Finland | CRR | N04 | 18,420,558 |
| Bn-A04-p16608584 | T:T | C:C | C:T | T:C | 0 | 2 | 1 | 1 | Finland | CRR | N04 | 18,548,589 |
| Bn-A04-p17358519 | C:C | T:T | C:T | T:C | 0 | 2 | 1 | 1 | Finland | CRR | N04 | 19,307,982 |
| Bn-A04-p17613869 | C:C | T:T | C:T | T:C | 0 | 2 | 1 | 1 | Finland | CRR | N04 | 19,579,676 |
| Bn-A04-p18562244 | G:G | T:T | G:T | T:G | 0 | 2 | 1 | 1 | Finland | CRR | N04 | 20,563,734 |
| Bn_N8_Norway_p10651892 | T:T | G:G | T:G | G:T | 0 | 2 | 1 | 1 | Norway | CRR | N08 | 10,651,892 |
| Bn_N8_Norway_p10661593 | G:G | C:C | G:C | C:G | 0 | 2 | 1 | 1 | Norway | CRR | N08 | 10,661,594 |
| Bn_N8_Norway_p10799175 | T:T | C:C | T:C | C:T | 0 | 2 | 1 | 1 | Norway | CRR | N08 | 10,799,176 |
| Bn_N8_Norway_p11126069 | T:T | A:A | T:A | A:T | 0 | 2 | 1 | 1 | Norway | CRR | N08 | 11,126,290 |
| Bn_N8_Norway_p11181573 | T:T | A:A | T:A | A:T | 0 | 2 | 1 | 1 | Norway | CRR | N08 | 11,181,573 |
| Bn_N8_Norway_p11468429 | T:T | A:A | T:A | A:T | 0 | 2 | 1 | 1 | Norway | CRR | N08 | 11,481,275 |
| Bn_N8_Norway_p11588240 | G:G | C:C | G:C | C:G | 0 | 2 | 1 | 1 | Norway | CRR | N08 | 11,588,240 |
| Bn_N8_Norway_p11730703 | C:C | T:T | C:T | T:C | 0 | 2 | 1 | 1 | Norway | CRR | N08 | 11,730,703 |
| Bn_N8_Norway_p11804795 | G:G | T:T | G:T | T:G | 0 | 2 | 1 | 1 | Norway | CRR | N08 | 11,804,795 |

TABLE 2
Resistance of Norway, Finland and Iceland to various strains/pathotypes of Clubroot

| Pathotype | Trait Location | | | | | | | | | | | | | | | |
|---|---|---|---|---|---|---|---|---|---|---|---|---|---|---|---|---|
| Designation | (Chromosome) | 2 | 3 | 3A | 3D | 5 | 6 | 8 | FTC5X* | LG1 | LG2 | LG3 | 3H | 2B | 5X | C4W* |
| Gen1 Resistance | | R | R | S | S | R | R | R | S | S | S | S | S | S | S | R |
| Norway | N08: DH12075 | R | R | R | R | R | R | R | R | R | R | R | R | R | R | S |
| | position 10,799,175 | | | | | | | | | | | | | | | |
| | to 11,730,703 | | | | | | | | | | | | | | | |
| Finland | N04: DH12075 | — | R | — | — | — | — | — | R | — | — | — | — | — | — | — |
| | position 16,884,111 | | | | | | | | | | | | | | | |
| | to 20,563,734 | | | | | | | | | | | | | | | |
| Iceland | N03: DH 12075 | R | R | R | R | R | R | R | R | R | R | R | R | R | R | S |
| | position 14,845,698 | | | | | | | | | | | | | | | |
| | to 15,791,893 | | | | | | | | | | | | | | | |

R = resistant
S = susceptible
— = untested
*= Unique to Cargill, not obtained from outside source

All publications, nucleotide and amino acid sequence identified by their accession nos., patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. A Brassica plant or a part thereof comprising:
all or part of at least one genomic sequence of a B. napus parent genome that confers clubroot resistance, wherein said genome sequence is selected from the group consisting of:
a) the genomic sequence on chromosome N08 between nucleotide positions 10,799,175 and 11,730,703;
b) the genomic sequence on chromosome N04 between nucleotide positions 16,884,111 and 20,563,734; and
c) the genomic sequence on chromosome N03 between nucleotide positions 14,845,698 to 15,791,893;
wherein said Brassica plant has resistance to one or more pathotypes of clubroot (CR).

2. The Brassica plant or the part thereof of claim 1, wherein said genomic sequence on chromosome N03 between nucleotide positions 14,845,698 to 15,791,893 comprises a single nucleotide polymorphism (SNP) at a position selected from the group consisting of 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; and 15,711,283.

3. The Brassica plant or the part thereof of claim 2, wherein said genomic sequence comprises at least five SNPs at five different positions selected from the group consisting of 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; and 15,711,283.

4. The Brassica plant or the part thereof of claim 2, wherein said genomic sequence comprises at least 10 SNPs at 10 different positions selected from the group consisting of 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; or 15,711,283.

5. The Brassica plant or the part thereof of claim 2, wherein said genomic sequence comprises at least 15 SNPs at 15 different positions selected from the group consisting of 10,708,311; 12,061,079; 12,279,028; 12,629,150; 13,606,968; 13,616,978; 13,787,184; 14,509,547; 14,641,501; 14,687,684; 14,693,565; 14,752,111; 14,805,303; 14,845,698; 15,359,664; 15,461,403; 15,694,613; 15,697,299; and 15,711,283.

6. The Brassica plant or the part thereof of claim 1, wherein said genomic sequence on chromosome N04 between nucleotide positions 16,884,111 to 20,563,734 comprises a SNP at a position selected from the group consisting of 16,548,548; 16,884,111; 18,420,558; 18,548,589; 19,307,982; 19,579,676 and/or 20,563,734.

7. The Brassica plant or the part thereof of claim 6, wherein said genomic sequence comprises at least two SNPs at two different positions selected from the group consisting of 16,548,548; 16,884,111; 18,420,558; 18,548,589; 19,307,982; 19,579,676 and 20,563,734.

8. The Brassica plant or the part thereof of claim 6, wherein said genomic sequence comprises at least five SNPs at five different positions selected from the group consisting of 16,548,548; 16,884,111; 18,420,558; 18,548,589; 19,307,982; 19,579,676 and 20,563,734.

9. The Brassica plant or the part thereof of claim 1, wherein said genomic sequence on chromosome N08 between nucleotide positions 10,799,175 to 11,730,703 comprises a SNP at a position selected from the group consisting of 10,651,892; 10,661,594; 10,799,176; 11,126,290; 11,181,573; 11,481,275; 11,588,240; 11,730,703 and 11,804,795.

10. The Brassica plant or the part thereof of claim 9, wherein said genomic sequence comprises at least two SNPs at two different positions selected from the group consisting of 10,651,892; 10,661,594; 10,799,176; 11,126,290; 11,181,573; 11,481,275; 11,588,240; 11,730,703 and 11,804,795.

11. The Brassica plant or the part thereof of claim 9, wherein said genomic sequence comprises at least five SNPs at five different positions selected from the group consisting of 10,651,892; 10,661,594; 10,799,176; 11,126,290; 11,181,573; 11,481,275; 11,588,240; 11,730,703 and 11,804,795.

12. The Brassica plant or the part thereof of claim 9, wherein said genomic sequence comprises at least nine SNPs at nine different positions selected from the group consisting of 10,651,892; 10,661,594; 10,799,176; 11,126,290; 11,181,573; 11,481,275; 11,588,240; 11,730,703 and 11,804,795.

13. The Brassica plant or the part thereof of claim 1, wherein said genomic sequence comprises all or part of said genomic sequence on chromosome N03 between nucleotide positions 14,845,698 and 15,791,893 and all or part of the genomic sequence on chromosome N04 between nucleotide positions 16,884,111 and 20,563,734.

14. The Brassica plant or the part thereof of claim 1, wherein said genomic sequence comprises all or part of said genomic sequence on chromosome N03 between nucleotide positions 14,845,698 and 15,791,893 and all or part of the genomic sequence on chromosome N08 between nucleotide positions 10,799,175 and 11,730,703.

15. The Brassica plant or the part thereof of anyone of claim 1, wherein said genomic sequence comprises all or part of said genomic sequence on chromosome N08 between nucleotide positions 10,799,175 and 11,730,703 and all or part of the genomic sequence on chromosome N04 between nucleotide positions 16,884,111 and 20,563,734.

16. The Brassica plant or the part thereof of claim 1, wherein said genomic sequence comprises from 25 to 50, 25 to 100, 50 to 200, 100 to 500, 250 to 1,000, 500 to 5,000, 2,000 to 10,000, 5,000 to 20,000, 10,000 to 100,000, 50,000 to 400,000, 25,000 to 1,000,000, 100,000 to 1,000,000, 200,000 to 1,000,000, or 500 to 1,000,000 contiguous nucleotides of the genomic sequence of said B. napus parent genome.

17. The Brassica plant or the part thereof of claim 1, wherein said plant is selected from the group consisting of Camelina sativa, Brassica napus, Brassica oleracea, Brassica juncea, Brassica nigra, Brassica rapa, and Brassica carinata.

18. The Brassica plant or the part thereof of claim 17, wherein said plant is selected from the group consisting of Brassica napus, Brassica rapa, and Brassica juncea.

19. The Brassica plant or the part thereof of claim 1, wherein said plant is tolerant of an herbicide.

20. The Brassica plant or the part thereof of claim 19, wherein said herbicide is selected from the group consisting of imidazolinone, dicamba, cyclohexanedione, sulfonylurea, glyphosate, glufosinate, phenoxy propionic acid, L-phosphinothricin, triazine, and benzonitrile.

21. The Brassica plant or the part thereof of claim 1, wherein said plant is resistant to one or more clubroot pathotypes selected from the group consisting of 2, 3, 3A, 3D, 5, 6, 8, FTC5X, LG1, LG2, LG3, 3H, 2B, 5X, C4W.
